(19) United States
(12) Patent Application Publication
(10) Pub. No.: US 2025/0064580 A1
(43) Pub. Date: Feb. 27, 2025

(54) SUTURELESS ANASTOMOTIC CONNECTION DEVICE

(71) Applicants: Robert DOWLING, University Park, PA (US); THE PENN STATE RESEARCH FOUNDATION, University Park, PA (US)

(72) Inventors: Robert DOWLING, University Park, PA (US); William S. PIERCE, University Park, PA (US); Gerson ROSENBERG, University Park, PA (US)

(21) Appl. No.: 18/721,082

(22) PCT Filed: Dec. 16, 2022
(86) PCT No.: PCT/US22/53195

§ 371 Date: Jun. 17, 2024

Related U.S. Application Data
(60) Provisional application No. 63/291,063, filed on Dec. 17, 2021.

(51) Int. Cl. A61F 2/24

(52) U.S. Cl. CPC A61F 2/2409; A61F 2/2418; A61F 2220/0008

(57) ABSTRACT

A prosthetic anastomosis fixation device comprising an inner support structure and an outer support structure positioned around the inner support structure and coupled to a prosthetic device, wherein the inner support structure comprises an expandable structure radially movable between an unexpanded to an expanded configuration, wherein in the expanded configuration, the inner support structure provides a radially outward force toward an inner surface of the outer support structure, wherein the inner and outer support structures are sized and configured to receive a portion of a biological conduit therebetween such that in the expanded configuration, the portion of a biological conduit is fixedly secured between the inner surface of the outer support structure and an outer surface of the inner support structure.

### CROSS-REFERENCE TO RELATED APPLICATIONS

This application claims the benefit of U.S. Provisional Application No. 63/291,063 filed, Dec. 17, 2021, the contents of which are incorporated herein by reference in their entirety.

### TECHNICAL FIELD

This disclosure relates to anastomosis devices for connecting native blood vessels to vascular grafts and other prosthetic devices.

### BACKGROUND

The resection and replacement of the aorta for aneurysmal disease and dissection is a common operation in which the use of handsewn anastomosis to connect vascular grafts to native blood vessels remains the standard of care. In the dissection of the ascending aorta, replacement is required as the mortality with non-operative therapy is very high (1% mortality/hour). Though the exact incidence of replacement of the aorta for dissection is not known population-based studies suggest an incidence in the US between 30,000 to 80,000 cases annually. The dissection disrupts the normal tissue integrity and makes the creation of standard handsewn anastomosis among the most difficult procedures in cardiac surgery. As a result, time to create this anastomosis is often long and suture line bleeding is common and can be very difficult to manage. Additionally, these operations require a period of hypothermic circulatory arrest to perform the distal aortic anastomosis. The time to create the anastomosis often results in significant periods of circulatory arrest with longer periods of circulatory arrest being associated with increased neurologic events and end-organ dysfunction.

Elective resection of aortic aneurysms is also a common operation in which the use of handsewn anastomosis is typically used. The exact number of elective resections of aortic aneurysms is unknown but estimates from the Society of Thoracic Surgeons suggest between 15,000 and 20,000 cases per year in the US. Likely a similar number of cases are performed in the EU. These patients often have thin-walled aortas and require periods of hypothermic circulatory arrest. Creating a fast, hemostatic anastomosis with a device as described herein that eliminates the use of needles and sutures will prevent issues related to fragile native tissue and abrogate issue related to prolonged times to create an anastomosis. Moreover, this approach could potentially improve patient outcomes by allowing surgeons to safely increase the extent of aortic resection. Up to 30% of patients that have resection of their ascending aorta will require an intervention for progressive aneurysmal disease of their aortic arch. A rapid and effective anastomotic device as described herein will allow surgeons to extend the resection to include the aortic arch followed by a rapid secure anastomosis to the proximal descending thoracic aorta. Rapid anastomoses to the arch branch vessels can then be performed eliminating concerns for progression of aortic arch disease.

Heart failure is another example procedure where a handsewn anastomosis is used to connect the native atrium to the artificial heart. The incidence of heart failure continues to increase while the prognosis remains poor, and few options are available for patients who fail medical therapy. Heart transplantation is limited to fewer than 6,000 procedures per year globally. The National Institutes of Health continues to identify the need for improved mechanical circulatory support (MCS) devices and has estimated that up to 175,000 patients could immediately benefit from MCS. Current generation left ventricular assist devices (LVADs) have improved outcomes but are still associated with significant morbidity a including a high rate of stroke (8% at 1 year) and mortality (5 year survival of 46%). Importantly, continuous flow total artificial hearts (TAH) has been developed, however, a major issue related TAH implantation is the extent of the operative therapy. Creation of the anastomoses of the native atrium to the atrial cuff of the TAH is particularly challenging due to thin atrial tissue with frequent tears of the tissue and/or needle hole bleeding. A double suture line, which is time consuming, is often used to avoid both bleeding and air entrainment through the suture line with the potential for cerebral air emboli. Development of a TAH atrial cuff, as described herein, that can be rapidly anastomosed to the native atrial tissue and provides improved hemostasis will: (1) decrease the complexity of the operation, (2) decrease intraoperative and perioperative bleeding and, (3) decrease the time on cardiopulmonary bypass. Longer times on cardiopulmonary bypass increase both operative morbidity and mortality.

In summary, a need in the art exists for a quickly and easily implanted, sutureless anastomosis for connecting native blood vessels to vascular grafts, total artificial heart devices, or other biological conduit such as a bile duct, ureter, and/or fallopian tube.

### SUMMARY

Certain examples of the present disclosure provide sutureless anastomosis fixation device.

A prosthetic anastomosis fixation device disclosed herein comprises: an inner support structure, an outer support structure positioned around the inner support structure and coupled to a prosthetic device, wherein the inner support structure comprises an expandable structure radially movable between an unexpanded to an expanded configuration, wherein in the expanded configuration, the inner support structure provides a radially outward force toward an inner surface of the outer support structure, and wherein the inner and outer support structures are sized and configured to receive a portion of a patient's blood vessel therebetween such that in the expanded configuration, the portion of a patient's blood vessel is fixedly secured (e.g., circumferentially) between the inner surface of the outer support structure and an outer surface of the inner support structure.

A method of attaching a prosthetic anastomosis device to a patient's blood vessel using a fixation device disclosed herein comprises: advancing a prosthetic fixation device to a treatment site at an opening in a patient's blood vessel, where the prosthetic fixation device includes an inner support structure comprising an expandable structure radially movable between an unexpanded to an expanded configuration, and an outer support structure positioned around the inner support structure and coupled to a distal end of a prosthetic device. The method further comprises advancing the inner support structure in the unexpanded configuration within the opening in the patient's blood vessel, positioning the outer support structure adjacent to an outer surface of the patient's blood vessel, and radially expanding the inner support structure toward the expanded configuration such that the inner support structure provides a radially outward force against an inner surface of the outer support structure thereby securing a portion of the patient's blood vessel between the inner surface of the outer support structure and an outer surface of the inner support structure.

A further implementation of a prosthetic anastomosis fixation device disclosed herein comprises: an inner support structure; an outer support structure positioned around the inner support structure and coupled to a prosthetic device; wherein the inner support structure comprises an expandable structure radially movable between an unexpanded to an expanded configuration, wherein in the expanded configuration, the inner support structure provides a radially outward force toward an inner surface of the outer support structure, wherein the inner and outer support structures are sized and configured to receive a portion of a biological conduit therebetween such that in the expanded configuration, the portion of a biological conduit is fixedly secured between the inner surface of the outer support structure and an outer surface of the inner support structure.

Another implementation of a prosthetic anastomosis fixation device disclosed herein comprises: an inner support structure coupled to a prosthetic device, and an outer support structure positioned around the inner support structure. Where the inner support structure comprises an expandable structure radially movable between an unexpanded to an expanded configuration. In the expanded configuration, the inner support structure provides a radially outward force toward an inner surface of the outer support structure, and where the inner and outer support structures are sized and configured to receive a portion of a patient's cardiac tissue (e.g., atrium, blood vessel) therebetween such that in the expanded configuration, the portion of a patient's blood vessel is fixedly secured between the inner surface of the outer support structure and an outer surface of the inner support structure.

Another method of attaching a prosthetic anastomosis device to a patient's blood vessel using a fixation device disclosed herein comprises: advancing a prosthetic fixation device to a treatment site at an opening in a patient's blood vessel, where the prosthetic fixation device includes an inner support structure coupled to the prosthetic device, the inner support structure comprising an expandable structure radially movable between an unexpanded to an expanded configuration, and an outer support structure configured to be positioned around the inner support structure. The method further comprises advancing the inner support structure in the unexpanded configuration within the opening in the patient's blood vessel, positioning the outer support structure adjacent to an outer surface of the patient's blood vessel, and radially expanding the inner support structure toward the expanded configuration such that the inner support structure provides a radially outward force against an inner surface of the outer support structure thereby securing a portion of the patient's blood vessel between the inner surface of the outer support structure and an outer surface of the inner support structure.

A further method of attaching a prosthetic anastomosis device to a patient's blood vessel using a fixation device disclosed herein comprises: advancing a prosthetic fixation device to a treatment site at an opening in a patient's blood vessel, where the prosthetic fixation device included an inner support structure coupled to the prosthetic device, the inner support structure comprising an expandable structure radially movable between an unexpanded to an expanded configuration; and an outer support structure configured to be positioned around the inner support structure. The method further comprises attaching the outer support structure to the blood vessel, advancing the inner support structure in the unexpanded configuration within the opening in the patient's blood vessel, positioning the outer support structure adjacent to an outer surface of the patient's blood vessel, and radially expanding the inner support structure toward the expanded configuration such that the inner support structure provides a radially outward force against an inner surface of the outer support structure thereby securing a portion of the patient's blood vessel between the inner surface of the outer support structure and an outer surface of the inner support structure.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 is a schematic drawing of a patient anatomy with a prosthetic fixation device according to some examples.

FIG. 2 is a perspective view of the example prosthetic fixation device of FIG. 1.

FIG. 3 is a partial perspective view of the example prosthetic fixation device of FIG. 1 in a closed/expanded configuration and including adjacent patient anatomy.

FIG. 4 is a perspective view of the example prosthetic fixation device of FIG. 1.

FIG. 5 is a partial cross-section view of the distal end of the example prosthetic fixation device of FIG. 1.

FIG. 6 is a partial cross-section view of the distal end of the example prosthetic fixation device of FIG. 1 including adjacent patient anatomy in an open/unexpanded configuration.

FIG. 7 is a partial cross-section view of the distal end of the example prosthetic fixation device of FIG. 6 including adjacent patient anatomy in a closed/expanded configuration.

FIG. 8 is a schematic drawing of a patient anatomy with a prosthetic fixation device according to another example.

Like reference symbols in the various drawings indicate like elements.

### DETAILED DESCRIPTION

Disclosed herein is a rapid, sutureless anastomosis that provides significant advantages over current handsewn techniques with the goal to improve patient outcomes. The present disclosure provides for devices and methods that allow for a rapid and hemostatic sutureless anastomoses in both cardiac and vascular surgery and other procedures involving the attachment of grafts or other materials to hollow viscus such as urologic or intestinal procedures. For example, the disclosed device and method can be used to provide anastomoses of grafts to the native heart, native blood vessels or any hollow viscus and can also benefit patients that require placement of a total artificial heart. As described herein, the anastomotic fixation device rapidly creates an anastomosis without any tissue penetration through the native tissue in a method that is much quicker compared to traditional handsewn techniques. Moreover, the elimination of needle hole bleeding and decreased operative times will lead to improved patient outcomes.

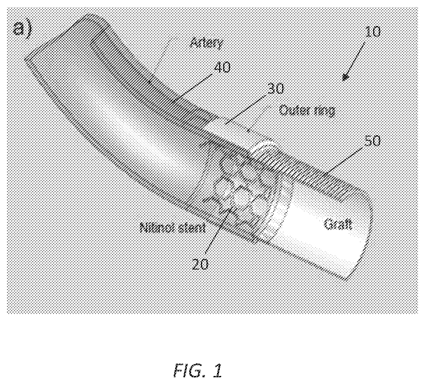

FIG. 1 illustrates a schematic drawing of a patient with an example prosthetic anastomosis fixation device 10. The prosthetic fixation device 10 includes an inner support structure 20 and an outer support structure 30 positioned circumferentially around the inner support structure 20. A prosthetic device 50 is coupled to the inner support structure 20 and/or the outer support structure 30. Example prosthetic devices 50 include a graft material and/or other biological conduit.

The inner and outer support structures 20, 30 are sized and configured to receive a portion of a patient's anatomy (e.g., blood vessel) therebetween such that the portion of a patient's anatomy 40 (e.g., blood vessel) is fixedly secured between the inner surface 32 of the outer support structure 30 and an outer surface 22 of the inner support structure 20. For example, the patient's blood vessel is secured circumferentially between the inner support structure 20 and the outer support structure 30.

The inner support structure 20 defines an annular generally ring-shaped structure with a central lumen 24 extending therethrough. As illustrated in FIGS. 1-7, the inner support structure 20 and outer support structure 30 are coupled to the prosthetic device 50 at their proximal ends 26, 36. The inner support structure 20 comprises an expandable structure that is radially movable between an unexpanded configuration (FIGS. 5, 6) and an expanded configuration (FIGS. 1, 3, 7).

As illustrated in FIGS. 5 and 6, in the unexpanded configuration, the diameter of the inner support structure 20 at the proximal end 26 is greater than the diameter of the inner support structure 20 at the distal end 28. In the expanded configuration, the diameter of the inner support structure 20 at the distal end 28 corresponds to the diameter at the proximal end 26. In the expanded configuration, the inner support structure 20 provides a radially outward force toward an inner surface 32 of the outer support structure 30, thereby fixing the patient's anatomy between the inner surface 32 of the outer support structure 30 and the outer surface 22 of the inner support structure 20. For example, in the expanded configuration, the inner support structure 20 is sized and configured to fit snugly on the inside of the patient's anatomy 40 (e.g., inner surface of the blood vessel). In some examples, the inner support structure 20 has an expanded diameter ranging from 1 mm to 100 mm, including exemplary values of 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm. In still further aspects, the diameter can have any value between any of the foregoing values. For example, the diameter can be between (and including) 15 mm to 50 mm, 18 mm to 36 mm, 50 mm to 55 mm, 50 mm to 100 mm.

As provided in FIGS. 1, 4 and 7, the axial length of the inner support structure 20 in the expanded configuration corresponds to the length of the outer support structure 30. Alternatively, the inner support structure 20, in the expanded configuration, can have a length that is greater or less than a length of the outer support structure 30. In some examples, the inner and outer support structures 20, 30 have a length ranging from 0.5 cm to 6.5 cm, including exemplary values of 0.5 cm, 1.0 cm, 1.5 cm, 2.0 cm, 2.5 cm, 3.0 cm, 3.5 cm, 4.0 cm, 4.5 cm, 5.0 cm, 5.5 cm, 6.0 cm, 6.5 cm. In still further aspects, the length of the inner and outer support structures 20, 30 have any value between any of the forgoing values. For example, the length can be between (and including) 2.0 cm to 6.0 cm.

The inner and outer support structures 20, 30 can have the same or varying thickness (measured in a radial direction between the inner and outer surface of the corresponding inner and outer support structures 20, 30). In some examples, the thickness of each of the inner and outer support structures 20, 30 ranges from 0.5 mm to 3 mm, including exemplary values of 0.5 mm, 1 mm, 1.5 mm, 2.0 mm, 2.5 mm, 3.0 mm. In still further aspects, the thickness of the inner and outer support structures 20, 30 have any value between any of the foregoing values. For example, the thickness can be between (and including) 1.0 mm and 2.5 mm. In some examples, the inner support structure 20 comprises an expandable stent coupled at the proximal end to the prosthetic device 50. In general, because the stent-like proximal end 26 of the inner support structure 20 is coupled to the prosthetic device 50, as the inner support structure 20 expands the diameter of the distal end 28 of the stent-like inner member expands while the diameter of the proximal end of the stent-line inner member remains constant. In some examples, in the unexpanded configuration the distal end portion of the stent-like inner member is more crimped and has a small diameter than a proximal end portion of the stent-like inner member so that the patient anatomy 40 can be advanced between the inner and outer support structures 20, 30. The distal end portion of the stent-like inner member is then expanded from the unexpanded configuration to the expanded configuration. As a result, the patient anatomy 40 is fixed between the stent-like inner member (inner support structure 20) and the outer support structure 30.

The inner support structure 20 can include an exposed/uncovered expandable stent such that the inner support structure 20 does not include any covering and/or coating over all or a portion of it's inner or outer surface. For example, the inner support structure 20 can include a bare metal expandable stent. In further examples, the inner support structure 20 includes a covering material (e.g., fabric), for example, the inner support structure 20 comprises a cloth covered expandable stent. The covering material prevents damage to the patient anatomy 40 and also provides for increased grip/resistance between the inner support structure 20 and the patient's anatomy 40 when securing the fixation device 10.

As described above, the prosthetic fixation device 10 includes an outer support structure 30 positioned circumferentially around the inner support structure 20. As illustrated in FIGS. 1-7, the outer support structure 30 defines a generally annular ring-shaped structure with a central lumen 34 extending therethrough.

As described above, the prosthetic device 50 is coupled to the inner support structure 20 and/or the outer support structure 30. In some examples, the prosthetic device 50 is coupled to both the inner support structure 20 and the outer support structure 30. In another example, the prosthetic device 50 is coupled to the outer support structure 30 and is separate from the inner support structure 20. In this example, the outer support structure 30 (and prosthetic device 50) is positioned adjacent the corresponding portion of the patient anatomy 40. Once positioned, the inner support structure 20 is advanced within the central lumen of the patient anatomy/blood vessel and expanded to secure the anatomy between the inner and outer support structures 20, 30.

In a further example, the prosthetic device 50 is coupled to the inner support structure 20 and is separate from the outer support structure 30. In this example, the inner support structure 20 (and the prosthetic device) is positioned adjacent the corresponding portion of the patient anatomy 40. Once the inner support structure 20 is positioned, the outer support structure 20 is separately advanced and positioned over the patient anatomy/blood vessel and the inner support structure is expanded to secure the anatomy 40 between the inner and outer support structures 20, 30. The outer support structure 30 can be separately coupled to the outer surface of the patient's anatomy 40. For example, the outer support structure 30 is coupled to the outer surface of the blood vessel, e.g., atrial tissue. In some examples, the outer support structure 30 is coupled to the blood vessel using a mechanical and/or chemical fastener (e.g., an adhesive such as BioGlue™ by CryoLife). In some examples, the outer support structure 30 includes a material (e.g., felt material) coupled to the blood vessel atrial tissue using an adhesive. In an example fixation device 10, the inner surface 32 of the outer support structure 30 includes a textured surface and/or coating for improving grip between the fixation device 10 and the patient anatomy 40. The textured surface and/or coating can also allow for tissue ingrowth between the outer support structure 30 and the patient's anatomy. It is contemplated that the outer surface 22 of the inner support structure 20 can also include a textured surface and/or coating for improving grip with the patient anatomy 40 and/or allowing tissue ingrowth between the inner support structure 20 and the patient anatomy 40. Example textured surfaces include a flocked surface, laser etched surface, a texture material deposited, an adhesive deposited on the surface of the inner and/or outer support structures 20, 30, and combinations thereof.

The outer support structure 30 is sized and configured to fit snugly on the outside of the patient's anatomy, e.g., circumferentially around the outer surface of a patient's blood vessel. In some examples, the outer support structure 30 does not provide a radial inward force and the patient anatomy 40 is secured to the outer support structure 30 by the radially outward force of the inner support structure 20. In other examples, the outer support structure 30 provides a radially inward force for compressing the patient's anatomy 40 between the inner and outer support structures 20, 30. In further examples, both the inner support structure 20 provides an outward force and the outer support structure 30 provides an inward force for securing the patient's anatomy 40 between the adjacent layers of the fixation device 10.

The outer support structure 30 is sized and configured to fit snugly on the outer surface of the patient's anatomy 40 (e.g., outer surface of the blood vessel). In some examples, the outer support structure 30 has a diameter ranging from 1 mm to 100 mm, including exemplary values of 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm. In still further aspects, the diameter can have any value between any of the foregoing values. For example, the diameter can be between (and including) 15 mm to 50 mm, 50 mm to 100 mm.

Though constructed to fit snugly around the patient's anatomy (e.g., blood vessel), the outer support structure 30 can be constructed from a material that allows the outer support structure 30 it to be removed and/or repositioned over the inner support structure 20. For example, as described below, the outer support structure 30 can be constructed from a polymer material, including an elastomeric polymer material, configured to stretch to a larger diameter and contract back towards the initial, unexpanded diameter. The outer support structure 30 can further include weakening structure, such as score lines and/or etchings, that allow the outer support structure 30 to tear or stretch along the weakened structure. The outer support structure 30 can include multiple weakening structures spaced around the circumference of the outer support structure 30. In use, the outer support structure 30 is positioned over the patient's anatomy 40 at a location corresponding to the inner support structure 20. If the inner and/or outer support structure 20, 30 need to be repositioned or removed, the physician can tear or stretch the outer support structure 30 along the weakening structure/score line and remove the outer support structure 30 from the patient anatomy 40.

It is contemplated that the fixation device 10 includes fixation structure (i.e., inner and outer support structures 20, 30) coupled to both ends of the prosthetic device 50 so that the fixation device 10 can be used to join adjacent portions of patient anatomy, e.g., linking a prosthetic device between adjacent segments of a patient's blood vessel.

For example, the fixation device 10 will include a first inner support structure 20a and outer support structure 30a coupled to at the proximal end 52 of the prosthetic device 50, and a second inner support structure 20b and outer support structure 30b at the distal end 53 of the prosthetic device 50. The second inner support structure 20b and outer support structure 30b can include similar design and function as the first inner support structure 20a and outer support structure 30a. For example, the second inner support structure 20b can include an expandable structure (e.g., a radially expanding stent) radially movable between an unexpanded to an expanded configuration. The second outer support structure 30b is positioned circumferentially around the second inner support structure 20b. The distal end 54 of the prosthetic device 50 can be coupled to either the second inner support structure 20b and/or the second outer support structure 30b. Like the fixation structure at the proximal end 52 of the prosthetic device 50, the second inner and outer support structures 20b, 30b are sized and configured to receive a second portion of a patient's anatomy 40 (e.g., a second opening in the patient's blood vessel) therebetween such that the portion of a patient's anatomy 40 (e.g., blood vessel) is fixedly secured between the inner and outer support structures 20b, 30b. For example, in the expanded configuration, the second inner support structure 20b provides a radially outward force toward an inner surface of the second outer support structure 30b fixing the second opening of the patient's anatomy 40 therebetween.

In general, the inner support structure 20 and outer support structure 30 are constructed from a biologically inert material. For example, the inner support structure 20 and/or outer support structure 30 is constructed from at least one of a metal (e.g., stainless steel, nitinol) and a polymer (e.g., polyethylene, Teflon®). In some examples, the outer support structure 30 is constructed from a felt material stiffened with glue.

In some examples, the inner support structure 20 is constructed from a magnetic material and/or includes magnetic elements that are magnetically attracted to the outer support structure 30. Alternatively, the outer support structure 30 can be constructed from a magnetic material and/or includes magnetic elements that are magnetically attracted to the inner support structure 20.

As described above, example prosthetic devices 50 include a graft material and/or other prosthetic biological conduit. In certain examples, the prosthetic device 50 comprises a vascular graft. The prosthetic device 50 is composed of a biocompatible synthetic material. Example biocompatible synthetic materials include polytetrafluoroethylene (PTFE), polyester (e.g., Dacron®, Gortex®), silk fibroin, polyurethane, and/or any other material known in the art that is suitable as a replacement for a biological conduit.

In some example anastomosis fixation devices 10, the prosthetic device 50 is impregnated with a material for promoting sealing and/or preventing infection. For example, the impregnation material can include a sealant for promoting sealing between the patient's vascular structure and the prosthetic device 50 (e.g., gelatin, collagen). Additionally/alternatively, the impregnation material can include an additive that inhibits bacterial infection (e.g., antibiotic, antiseptic). In certain examples, the prosthetic device 50 is a gelatin-impregnated woven polyester vascular graft.

The use of the example sutureless anastomotic fixation device 10 for aortic and other vascular surgeries is described below. As described above, the use of the anastomotic fixation devices described herein allows for a rapid, hemostatic anastomotic techniques that will improve outcomes in complex operations such as the dissection of the ascending aorta and the resection of aortic aneurysms. The target patient anatomy comprises a patient's blood vessel including, for example, an arterial segment, a venous segment, and/or an atrium structure. It is further contemplated that the target patient anatomy may include any other biological conduit such as a bile duct, ureter, or fallopian tube. The method for positioning the fixation device 10 within the patient anatomy is described in reference to a patient blood vessel, however similar method may be used to connect a prosthetic device 50 to any other biological conduit.

An opening is first created in the patient's blood vessel, e.g., by transecting the patient's blood vessel. The diameter of the blood vessel can be measured to identify an inner and outer support structure 20, 30 having a diameter corresponding to the measured diameter of the patient's blood vessel.

The fixation device 10 and corresponding prosthetic device 50 is coupled to the patient's anatomy by advancing the fixation device 10 to a treatment site at the opening in the patient's blood vessel. The inner support structure 20 is advanced in an unexpanded configuration within the opening in the patient's blood vessel. The outer support structure 30 is positioned adjacent an outer surface of the patient's blood vessel (adjacent the opening) at a location corresponding to the to the inner support structure 20.

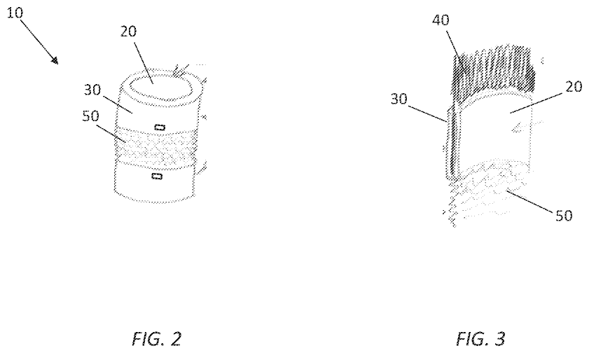

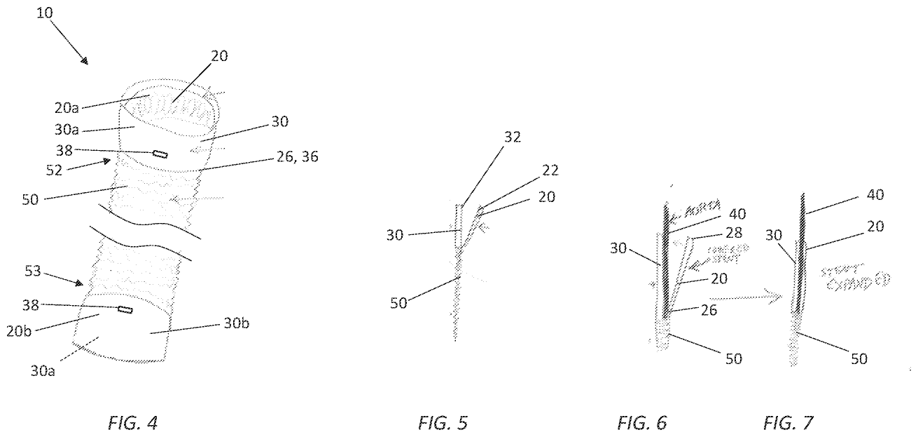

In some examples, traction stitches are used to position the fixation device 10. For example, traction stitches are placed in the patient's blood vessel, e.g., single or multiple stitches placed at various circumferential positions around the blood vessel. As illustrated in FIGS. 2 and 4, the outer support structure 30 includes a widow 38 extending from the outer to the inner surface of the outer support structure 30. The outer support structure 30 can include a single window 38 or a plurality of windows 38 spaced circumferentially around the outer support structure 30. In some examples, the window 38 comprises a circular or rectilinear shaped opening. In further examples, the window 38 comprises a longitudinally extending open slot extending from the end of the outer support structure 30 toward the prosthetic device 50. Positioning the inner support structure 20 and/or outer support structure 30 within the opening in the patient's blood vessel includes positioning or otherwise seating the traction stitch(es) within the window(s) 38. For example, when the blood vessel comprises the aorta, the traction stitches are placed on the aorta to ensure the aorta is well seated into the inner and outer support structures 20, 30. The traction stitches can be removed after the fixation device 10 has been secured to the patient's blood vessel.

Once the inner support structure 20 and outer support structure 30 are positioned, the inner support structure 20 is then radially expanded toward an expanded configuration such that the inner support structure 20 provides a radially outward force against an inner surface 32 of the outer support structure 30. As a result, a portion of the patient's blood vessel is secured between the inner surface 32 of the outer support structure 30 and an outer surface 22 of the inner support structure 20. In some examples, the outer support structure 30 includes a textured inner surface that grips the outer surface of the patient's blood vessel.

In an example where the blood vessel comprises an aorta, positioning the outer support structure 30 adjacent an outer surface of the patient's blood vessel includes positioning the inner surface 32 of the outer support structure 30 adjacent the adventitia of the aorta. Radially expanding the inner support structure 20 secures the aorta tissue between the inner support structure 20 and the outer support structure 30.

As described above, the fixation device 10 includes fixation structure (inner and outer support structure 20, 30) to both ends of the prosthetic device 50 so that the fixation device 10 can be used to join adjacent portions of patient anatomy, i.e., linking the prosthetic device 50 between adjacent segments of the patient's blood vessel. Accordingly, the fixation device 10 includes a first inner and outer support structure 20a, 30a is provided at the proximal end 52 of the prosthetic device 50 and a second inner and outer ring 20b, 30b is provided at the distal end 54 of the prosthetic device 50. The fixation structure at the proximal end 52 is coupled first to the patient anatomy as described above. The fixation structure at the distal end 54 of the prosthetic device 50 is then coupled to the patient's blood vessel by advancing the second inner support structure 20b in the unexpanded configuration within a second opening in the patient's blood vessel 40. The second outer support structure 30b is positioned adjacent an outer surface of the patient's blood vessel. The second inner support structure 20b is then radially expanded toward the expanded configuration such that the second inner support structure 20b provides a radially outward force against an inner surface 32 of the second outer support structure 30b. As a result, a second portion of the patient's blood vessel is secured between the inner surface 32 of the second outer support structure 30b and an outer surface 22 of the second inner support structure 20b.

The inner support structure 20a, 20b is radially expanded manually against the inner surface of the patient's blood vessel. In some examples, radially expanding the inner support structure 20a, 20b includes positioning a balloon expansion device within a central lumen of the inner support structure 20a, 20b and inflating the balloon to expand the inner support structure 20a, 20b. After the inner support structure 20a, 20b is secured against the blood vessel and the blood vessel secured against the outer support structure 30a, 30b, the balloon expansion device is deflated and moved away from the inner support structure 20a, 20b and the patient's blood vessel. In some examples, the balloon expansion device is coupled to the inner support structure 20a, 20b and/or the outer support structure 30a, 30b. For example, when coupled to the inner and/or outer support structure 20, 30, when the support structure is positioned within the patient anatomy at the treatment site, the balloon expansion device is correspondingly positioned to expand the expand the inner support structure 20a, 20b. In a further example, the balloon expansion device is separate from the inner and/or outer support structures 20, 30.

In the example process, securing the portion of the patient's blood vessel between the outer support structure 30a, 30b and the inner support structure 20a, 20b creates a liquid-tight seal between the inner support structure 20a, 20b, the blood vessel, and the outer support structure 30a, 30b. The seal between the inner support structure 20a, 20b, the blood vessel, and the outer support structure 30a, 30b is tested by flowing fluid (e.g., saline, blood) through the prosthetic device 50. For example, a clamp upstream of the prosthetic device 50 is released and blood is allowed to flow through the fixation device 10/prosthetic device 50. If a leak at the fixation device is determined, i.e., a liquid-tight seal between the between the inner support structure 20a, 20b, the blood vessel, and the outer support structure 30a, 30b is not present, the clamp is reapplied and the inner support structure 20a, 20b is additionally expanded to increase the radially outward pressure applied by the inner support structure 20a, 20b towards the outer support structure 30a, 30b. For example, additional inflations of the balloon may be employed to further expand the inner support structure 20a, 20b to achieve complete hemostasis.

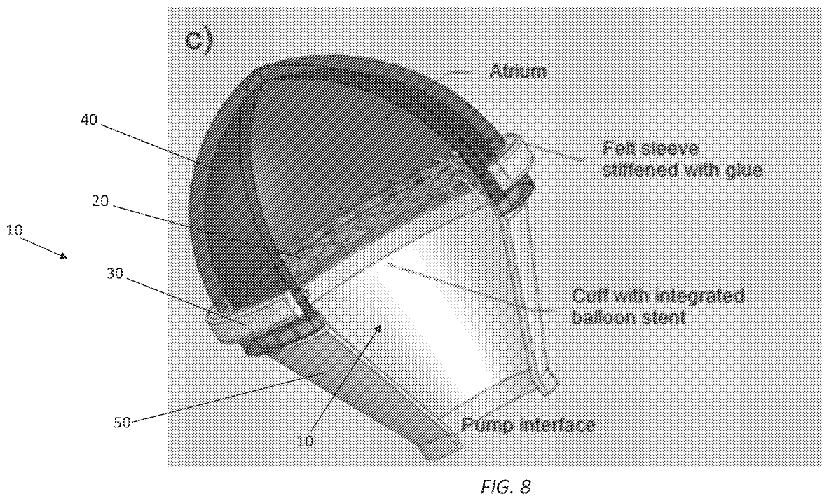

FIG. 8 illustrates a schematic drawing of a patient with a prosthetic fixation device 10 according to another example. In this example, the prosthetic device 50 includes at least one of a prosthetic heart valve, a heart assist pump, an artificial heart. For example, as provided in FIG. 8, the prosthetic device 50 is an atrial cuff of a total artificial heart.

The fixation device of FIG. 8 includes structure and materials similar to the fixation device of FIGS. 1-7. Like element numbers are used to identify like structure. The differences between the fixation device of FIGS. 1-7 and the device of FIG. 8 are provided in more detail below.

The fixation device 10 of FIG. 8 includes an inner support structure 20 coupled to a prosthetic device 50 (i.e., atrial cuff) and an outer support structure 30 positioned around the inner support structure 20. Similar to the device above, the inner support structure 20 comprises an expandable structure radially movable between an unexpanded to an expanded configuration. In the expanded configuration, the inner support structure 20 provides a radially outward force against the patient anatomy and toward an inner surface 32 of the outer support structure 30. The inner and outer support structures 20, 30 are sized and configured to receive a portion of a patient's cardiac tissue (e.g., atrium). As such, in the expanded configuration, the portion of a patient's atrium is fixedly secured between the inner surface 32 of the outer support structure 30 and an outer surface 22 of the inner support structure 22.

The inner support structure 20 and outer support structure 30 each have an annular shape including corresponding central lumen extending therethrough. In some examples, the cross-sectional shape of the central lumen of each of the inner and outer support structures 20, 30 corresponds to the cross-sectional shape of the atrium (e.g., the cross-sectional shape in the transverse plane).

In the expanded configuration, the inner support structure 20 is sized and configured to fit snugly inside the patient's atrium. The diameter/width of the inner support structure 20 ranges from 20 mm to 100 mm, including exemplary values of 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm. In still further aspects, the diameter/width can have any value between any of the foregoing values. For example, the diameter can be between (and including) 20 mm to 70 mm, 30 mm to 70 mm. As illustrated in FIG. 8, the length of the inner support structure 20 in the expanded configuration is greater than the length of the outer support structure 30. In other examples, the length of the inner support structure 20 in the expanded configuration corresponds to a length of the outer support structure 30.

The use of the example sutureless anastomotic fixation device 10 for aortic surgeries and implantation of total artificial hearts is described below. As described above, the use of the anastomotic fixation devices described herein allows for a rapid, hemostatic techniques for the creation of the anastomoses of the native atrium to the atrial cuff of a total artificial heart.

An opening is first created in the patient's atrium, e.g., the heart is removed to exposed the open atrium. The diameter and/or width of the opening is measured to identify inner and outer support structure 20, 30 having a corresponding diameter and/or width.

The fixation device 10 and corresponding prosthetic device 50 is coupled to the patient's atrium by advancing the fixation device 10 to a treatment site at an opening in the patient's atrium. The inner support structure 20 is advanced in an unexpanded configuration within the opening such that the outer surface 22 of the inner support structure 20 is adjacent an inner surface of the atrium. The outer support structure 30 is positioned adjacent an outer surface 32 of the atrium at a location corresponding to the to the inner support structure 20 such that the atrial tissue is positioned between the inner and outer support structures 20, 30.

In some examples, traction stitches are used to position the fixation device 10. For example, traction stitches are placed in the atrium, e.g., single or multiple stitches can be placed at various circumferential positions around the atrium. The outer support structure 30 includes a widow(s) 38 extending through the outer support structure 30. As described above, the outer support structure 30 can include a single window 38 or a plurality of windows 38 spaced circumferentially around the outer support structure 30. Positioning the inner support structure 20 and/or outer support structure 30 within the opening in the patient's atrium includes positioning or otherwise seating the traction stitches within the window(s) 38.

Once the inner support structure 20 and outer support structure 30 are positioned, the inner support structure 20 is then radially expanded toward an expanded configuration such that the inner support structure 20 provides a radially outward force against the atrium and the inner surface 32 of the outer support structure 30. As a result, a portion of the patient's atrium is secured between the inner surface 32 of the outer support structure 30 and an outer surface 22 of the inner support structure 20. In some examples, the inner and/or outer support structures 20, 30 include textured surfaces to improve grip with the atrium tissue.

In some examples, radially expanding the inner support structure 20 includes manually expanding the inner support structure 20 against an inner surface of the patient's atrium.

In some examples, radially expanding the inner support structure 20 includes positioning a balloon expansion device within a central lumen of the inner support structure 20 and inflating the balloon to expand the inner support structure 20. After the inner support structure 20 is secured against the atrium and the atrium is secured against the outer support structure 30, the balloon expansion device is deflated and removed from the inner support structure 20 and the patient's atrium.

In an example process, securing the portion of the patient's atrium between the outer support structure 30 and the inner support structure 20 creates a liquid-tight seal between the inner support structure 20, the atrium, and the outer support structure 30. The seal between the inner support structure 20, the atrium, and the outer support structure 30 is tested by flowing fluid through the prosthetic device 50. For example, an obturator is introduced into a central lumen of the atrial cuff. A foley catheter is advanced upstream of the prosthetic device and inflated to occlude blood flow through/from the pulmonary veins. Liquid (e.g., blood, saline solution) is provided into the central lumen of the atrial cuff and any leakage around the inner and outer support structures 20, 30 is determined.

If a leak at the fixation device is determined, i.e., there is not a liquid-tight seal between the between the inner support structure 20, the atrium, and the outer support structure 30, the inner support structure 20 is additionally expanded to increase the radially outward pressure applied by the inner support structure 20 towards the outer support structure 30. For example, additional inflations of the balloon may be employed to further expand the inner support structure 20 to achieve complete hemostasis.

Although several examples of the invention have been disclosed in the foregoing specification, it is understood by those skilled in the art that many modifications and other examples of the invention will come to mind to which the invention pertains, having the benefit of the teaching presented in the foregoing description and associated drawings. It is thus understood that the invention is not limited to the specific examples disclosed hereinabove and that many modifications and other examples are intended to be included within the scope of the appended claims. Moreover, although specific terms are employed herein, as well as in the claims which follow, they are used only in a generic and descriptive sense and not for the purposes of limiting the described invention nor the claims which follow. We, therefore, claim as our invention all that comes within the scope and spirit of these claims.

1. A prosthetic fixation device comprising:
an inner support structure;
an outer support structure positioned around the inner support structure and coupled to a prosthetic device;
wherein the inner support structure comprises an expandable structure radially movable between an unexpanded to an expanded configuration,
wherein in the expanded configuration, the inner support structure provides a radially outward force toward an inner surface of the outer support structure,
wherein the inner and outer support structures are sized and configured to receive a portion of a biological conduit therebetween such that in the expanded configuration, the portion of the biological conduit is fixedly secured between the inner surface of the outer support structure and an outer surface of the inner support structure.

2. The device of claim 1, further including:
a second inner support structure comprising an expandable structure radially movable between an unexpanded to an expanded configuration;
a second outer support structure positioned around the second inner support structure and coupled to a distal end of the prosthetic device;
wherein in the expanded configuration, the second inner support structure provides a radially outward force toward an inner surface of the second outer support structure,
wherein the second inner and outer support structures are sized and configured to receive a second portion of a biological conduit therebetween such that in the expanded configuration, the second portion of a biological conduit is fixedly secured between the inner surface of the second outer support structure and an outer surface of the second inner support structure.

3. The device of claim 1, wherein the outer support structure is coupled to the prosthetic device at a proximal end of the outer support structure,
wherein the inner support structure is coupled to the prosthetic device at a proximal end of the inner support structure.

4. The device of claim 3, wherein, in the unexpanded configuration, a proximal end diameter of the inner support structure is greater than a distal end diameter of the inner support structure.

5. The device of claim 1, wherein the inner support structure and the outer support structure each define an annular ring shape.

6. The device of claim 1, wherein the inner support structure is an expandable stent.

7. The device of claim 6, wherein a distal end portion of the expandable stent expands from an unexpanded configuration to an expanded configuration, wherein in the unexpanded configuration the distal end portion is more crimped than a proximal end portion of the expandable stent.

8. (canceled)

9. The device of claim 1, wherein the inner support structure comprises a cloth covered expandable stent.

10. (canceled)

11. The device of claim 1, wherein the inner support structure is magnetically attracted to the outer support structure.

12. (canceled)

13. The device of claim 1, wherein, in the expanded configuration, the inner support structure is sized and configured to fit snugly on an inside of the biological conduit,
wherein the outer support structure is sized and configured to fit snugly on an outside of the biological conduit.

14. The device of claim 1, wherein a length of the inner support structure in the expanded configuration corresponds to a length of the outer support structure.

15. The device of claim 1, wherein a length of the inner support structure is greater than or less than a length of the outer support structure.

16. (canceled)

17. The device of claim 1, wherein the inner surface of the outer support structure includes a textured surface for gripping the biological conduit.

18. The device of claim 17, wherein the textured surface includes at least one of a flocked surface, laser etched surface, a texture material deposited on the inner surface of the outer support structure, or an adhesive deposited on the inner surface of the outer support structure.

19. (canceled)

20. (canceled)

21. (canceled)

22. (canceled)

23. The device of claim 1, wherein the prosthetic device includes at least one of a graft material, a biological conduit, a vascular graft, a prosthetic heart valve, a heart assist pump, or an artificial heart.

24. (canceled)

25. (canceled)

26. The device of claim 1, wherein the prosthetic device is impregnated with an impregnation material including at least one of a sealant for promoting sealing between a patient's vascular structure and the prosthetic device or an additive for inhibiting bacterial infection.

27. (canceled)

28. A method of attaching a prosthetic device to a biological conduit, the method comprising:
advancing a prosthetic fixation device to an opening in a biological conduit, the prosthetic fixation device including:
an inner support structure comprising an expandable structure radially movable between an unexpanded to an expanded configuration; and
an outer support structure positioned around the inner support structure and coupled to a distal end of a prosthetic device;
advancing the inner support structure in the unexpanded configuration within the opening in the biological conduit;
positioning the outer support structure adjacent an outer surface of the biological conduit; and
radially expanding the inner support structure toward the expanded configuration such that the inner support structure provides a radially outward force against an inner surface of the outer support structure thereby securing a portion of the biological conduit between the inner surface of the outer support structure and an outer surface of the inner support structure.

29. The method of claim 28, wherein the prosthetic fixation device further includes:
a second inner support structure comprising an expandable structure radially movable between an unexpanded to an expanded configuration;
a second outer support structure positioned around the second inner support structure and coupled to a distal end of the prosthetic device;
wherein the method of further includes:
advancing the second inner support structure in the unexpanded configuration within a second opening in the biological conduit;
positioning the second outer support structure adjacent an outer surface of the biological conduit;
radially expanding the second inner support structure toward the expanded configuration such that the second inner support structure provides a radially outward force against an inner surface of the second outer support structure thereby securing a second portion of the biological conduit between the inner surface of the second outer support structure and an outer surface of the second inner support structure.

30. (canceled)

31. The method of claim 29, wherein radially expanding the inner support structure includes positioning a balloon expansion device within a central lumen of the inner support structure and inflating the balloon to expand the inner support structure,
wherein, after the inner support structure is secured against the biological conduit and the biological conduit secured against the outer support structure, the balloon expansion device is deflated and moved away from the inner support structure and the patient's blood vessel biological conduit.

32. The method of claim 28, wherein securing the portion of the biological conduit between the outer support structure and the inner support structure creates a liquid-tight seal between the inner support structure, the biological conduit, and the outer support structure,
wherein seal between the inner support structure, the biological conduit, and the outer support structure is tested by flowing fluid through the prosthetic device,
wherein if a liquid-tight seal between the between the inner support structure, the biological conduit, and the outer support structure is not determined, the inner support structure is additionally expanded.

33-81. (canceled)
